(19) United States
(12) Patent Application Publication
(10) Pub. No.: US 2006/0026040 A1
(43) Pub. Date: Feb. 2, 2006

(54) System and method for providing remote analysis of medical data

(76) Inventors: Anthony P. Reeves, Ithaca, NY (US); David Yankelevitz, Brooklyn, NY (US); Claudia Henschke, New York, NY (US)

Correspondence Address: Fay Kaplun & Marcin, LLP, Suite 702, 150 Broadway, New York, NY, 10038, US

(21) Appl. No.: 10/901,316

(22) Filed: Jul. 28, 2004

(51) Int. Cl. A61B 5/00

(52) U.S. Cl. 705/3; 382/128

(57) ABSTRACT

A system and method for providing remote analysis of medical imaging data. The system may include a remote server with a database and a plurality of imaging data analyzing modules. The remote server receives, via a communications network, medical data from a medical facility and stores the medical data in the database. The medical data includes at least one identifier and imaging data generated at the medical facility. The server receives a request for analysis of the imaging data from a user. In response to the user request, the server controls one of the plurality of modules to analyze the imaging data to generate output data which is stored in the database. The server provides at least one of the imaging data and the output data to a remote requester based on the at least one identifier.

### FIELD OF THE INVENTION

The present invention relates generally to analysis of medical data, and, more particularly to analysis of medical data generated at a geographically remote medical facility and received at a data analysis facility.

### BACKGROUND INFORMATION

Conventional methods for analysis of medical imaging data are costly and complex. As medical imaging scans, such as radiology scans, have become more complex and detailed, the amount of data generated from a single patient's scans has increased tremendously. If performed without computing devices and modules as tools for analysis, analysis of medical imaging scan data has become increasingly burdensome and may delay the patient's diagnosis and/or treatment. Where the medical imaging scan is used for diagnosis of life-threatening illnesses, such detecting and measuring nodules for diagnosis of cancer, a delay may have serious consequences for the patient. In addition, processing such an increased amount of data requires a significant investment in dedicated equipment and software including, for example, high-resolution specialized display monitors and dedicated modules each equipped with a different set of software analysis tools for deciphering and diagnosing potential abnormalities. In addition, such modules require frequent and costly maintenance and updates. Because of the high cost involved with acquiring and maintaining these modules, they are generally found only at large and well-funded medical facilities.

Adding to the complexity of handling medical imaging data are recently propagated government regulations such as, for example, the Health Insurance Portability and Accountability Act of 1996 (HIPAA). The HIPAA imposes national standards for electronic health care transactions and national identifiers for providers, health plans, and employers. The HIPAA also mandates regulations for the security and privacy of health data. The present invention provides a system compatible with privacy requirements for handling the widespread use of electronic data interchange in health care.

### SUMMARY OF THE INVENTION

The present invention relates to a system and method for providing remote analysis of medical imaging data. The system may include a remote server with a database and a plurality of imaging data analyzing modules. The remote server receives, via a communications network, medical data from a medical facility and stores the medical data in the database. The medical data includes at least one identifier and imaging data generated at the medical facility. The server receives a request for analysis of the imaging data from a user. In response to the user request, the server controls one of the plurality of modules to analyze the imaging data to generate output data which is stored in the database. The server provides at least one of the imaging data and the output data to a remote requester based on the at least one identifier.

### BRIEF DESCRIPTION OF THE DRAWINGS

FIG. 1 shows an exemplary embodiment of a system for providing remote analysis of medical data according to the present invention;

FIG. 2 shows an exemplary embodiment of a method for providing remote analysis of medical data according to the present invention; and

FIG. 3 shows an exemplary embodiment of a medical device medical data record according to the present invention.

### DETAILED DESCRIPTION

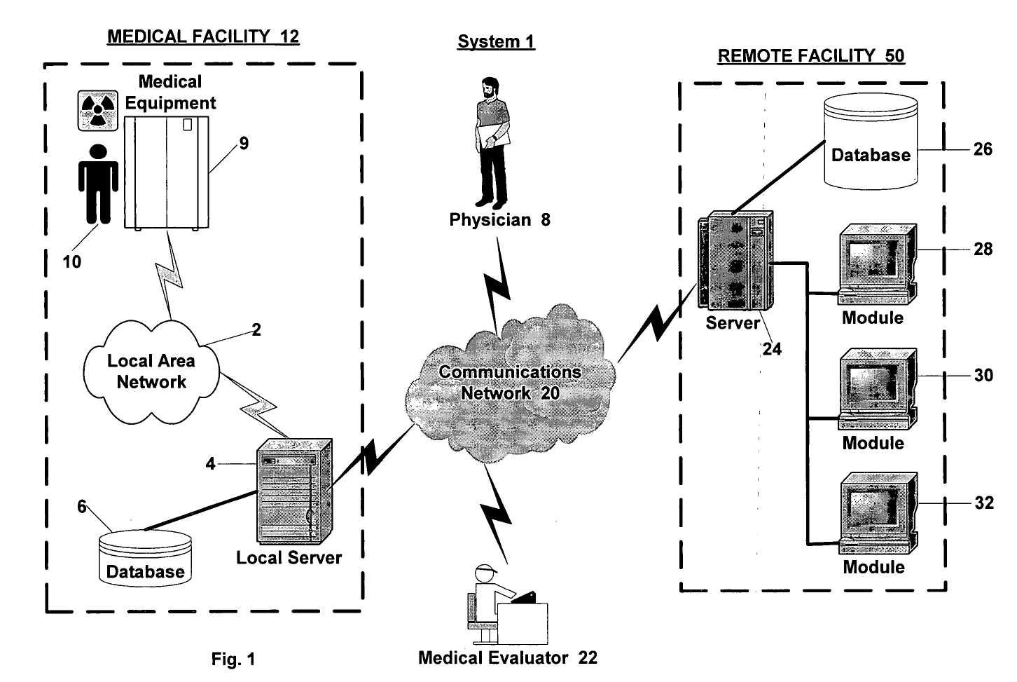

FIG. 1 shows an exemplary embodiment according to the present invention of a system 1 for providing remote analysis of medical data 102 of a patient 10. The medical data 102 may include, e.g., radiology data, radiology images, medical image data, pathology image data, digital images of medical data, photographic images, scanned images, molecular imaging data, medical genetic imaging data, etc. The medical data 102 may be collected/generated at a medical facility 12 and transmitted, via a communications network 20, to a remote facility 50 for analysis.

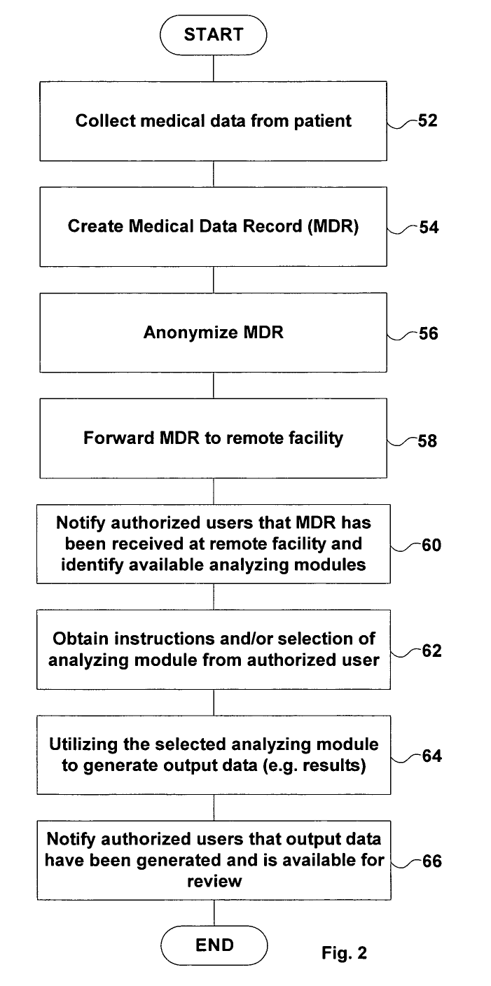

FIG. 2 shows an exemplary embodiment of the method according to the present invention. In step 52, the medical facility 12 collects the medical data 102 from the patient 10. In particular, the medical facility 12 may perform a medical procedure or analysis on the patient 10 using a medical device 9 to generate the medical data 102. The medical procedure may include, e.g., a Computerized Tomography (CT) scan, Magnetic Resonance Imaging (MRI), Positron Emission Technology (PET), X-Rays, Vascular Interventional and Angiogram/Angiography procedures, Ultrasound imaging, and similar procedures. In one exemplary embodiment of the present invention, the medical facility 12 may collect complex and detailed three-dimensional images of the patient's lungs utilizing a Computerized Tomograph (CT) Scan imager for detection and measurement of nodules for a lung cancer diagnosis.

In step 54, the medical data 102 is forwarded to a local server 4, via a local area network 2, for creation of a Medical Data Record (“MDR”) 100. In particular, the MDR 100 is generated by the local server 4 using the medical data 102 along with other data which is described below.

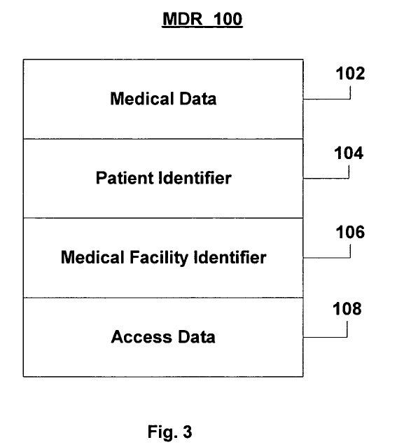

FIG. 3 shows an exemplary embodiment of the MDR 100. The MDR 100 may include, in addition to the medical data 102, a patient identifier 104, a medical facility identifier 106 and an access data 108 indicating access parameters for the medical data 102. The patient identifier 104 may include patient's personal information (e.g., name, address, social security number, etc.). The access data 108 provides data regarding varying degrees of access to the MDR 100. For example, the access data 108 includes a list of authorized users and corresponding level of access. As would be understood by those skilled in the art, the authorized user may include a medical evaluator 22 (e.g., a radiologist), a physician 8, and/or other user functionaries.

In step 56, the MDR 100 is modified in preparation for transmission to the remote facility 50. In particular, the local server 4, to preserve patient's confidentiality and comply with HIPAA requirements, modifies the patient's identifier 104. In one exemplary embodiment, the local server 4 may assign a randomly generated anonymous identifier. Then, the patient's personal information (e.g., name, address, social security number, etc.) is removed from the patient's identifier 104 and replaced with the anonymous identifier. The local server 4 may store the patient's personal information along with the corresponding anonymous identifier in the database 6. Once corresponding output data is received from the remote facility 50, the local server 4 is able to determine the corresponding patient's personal information using the anonymous identifier.

In step 58, the medical facility 12 forwards the modified MDR 100 to the remote facility 50 via the communications network 20 (e.g., the Internet, a Wide Area Network or another computer communications network). The remote facility 50 may be external and independent of the medical facility 12 and located anywhere in the world.

The remote facility 50 may include a server 24, a database 26 which stores the MDR 100 and a plurality of analyzing modules 28, 30, 32, etc. The remote facility 50 is generally separate and independent form the medical facility 12. The remote facility 50 is responsible for obtaining (e.g., purchasing, leasing, etc.) and maintaining the analyzing modules 28-32. Each of the analyzing modules 28-32 may perform a designated task of analyzing the medical data 102. Thus, the analyzing module 28-32 receives as input the medical data 102, analyzes the medical data 102 and generates the output data.

The analyzing module 28-32 may include, for example, tools that have been developed for a nodule detection on CXR and CT scans including computer algorithms that utilize high-resolution data more efficiently to improve performance. The analyzing modules 28-32 may also include a remote analysis of morphology features module, a growth assessment module, a volume determination module, a nodule detection module (e.g., for CXR or CT scans), an emphysema evaluation module, an airway evaluation module, a feature analysis module, a vascular analysis module, a two-dimensional analysis module, a three-dimensional analysis module, selected one dimensional approaches and a time dependent four-dimensional module.

In one exemplary embodiment, one or more modules may include a management system such as the ELCAP management system (EMS). The EMS is a web-based management tool which includes image storage and analysis components; it manages all aspects of patient scheduling, clinical information, transfer of images, and image interpretation. The EMS also includes the highest quality measuring tools available that allow for volumetric measurement of nodules. However, it will be understood that the invention is not so limited and that it provides a universal platform with capability to incorporate substantially any number or type of computer analysis modules as they become available.

In step 60, the medical facility 12 and/or the remote facility 50 may notify (e.g., phone, fax, email) predefined authorized users, as listed in the access data 108, that the MDR 100 has been transmitted to or received by the remote facility 50 and is available for further analysis. In addition, the remote facility 50 provides information to the authorized users regarding availability and functionality of the analyzing modules 28-32.

In step 62, the authorized users can access the remote facility 50, e.g., via the communications network 20, by providing an access code. The authorized user provides an indication to the remote server 24 as to which module (e.g., the analyzing module 30) is selected to utilize for analysis of the medical data 102.

In step 64, the remote server 24 instructs the selected analyzing module 30 to perform the analysis of the medical data 102. The analyzing module 30 generates output data which is stored in the database 26. For example, the medical facility may forward the MDR that contains CT scan images of a patient's lungs to the remote facility for detection and measurement of nodules for lung cancer diagnosis. Before performing any manual review of the images, a radiologist may access the remote facility and select a particular analyzing module. The module analyzes the images, generates reports, flags certain images or a particular nodules for the radiologist, etc. These results may assists the radiologist in reviewing and issuing of a report.

In step 66, the authorized users are notified that the output data had been generated and is available for access. Alternatively, or in addition, the output data is transmitted to the medical facility 12. The medical facility 12 then using the anonymous identifier, determines the patient's personal information and stores the output data in corresponding patient's record.

One of the advantages of the present invention is that the medical facility 12 or any authorized user does not have to purchase and maintain the analyzing modules. On other hand, the analyzing modules 28-32 are available for analyzes when needed. For example, the analyzing modules 28-32 may be utilized on a pay-per-use basis or any other payment model desired. For example, monthly payments for usage up to a threshold level with pay-per-use charges for use in excess of the threshold level. For the pay-per-usage model, each analysis of the medical data 102 results in a predefined charge directly attributable to the corresponding patient 10, medical facility 12, physician 8 or radiologist 22 and, therefore, billable thereto or to a corresponding medical insurance company, etc.

In addition, once the medical data 102 and the results have been stored in the database 26, they may be held in the database 26 indefinitely to provide immediate access to all authorized users. For example, if the patient 10 is admitted by a further medical facility and a further medical procedure is performed, a physician at the further medical facility may access the data by contacting the remote facility 50 (e.g., also based on pay-per-access basis) to view the prior medical data and related results.

While specific embodiments of the invention have been illustrated and described herein, it is realized that numerous modifications and changes will occur to those skilled in the art. It is therefore to be understood that the appended claims are intended to cover all such modifications and changes as fall within the true spirit and scope of the invention.

What is claimed is:

1. A method, comprising the steps of:
receiving, at a data analysis facility, medical data transmitted, via a communications network, from a medical facility, the medical data including at least one identifier and imaging data generated at the medical facility, the data analysis facility including a computing arrangement with a database and a plurality of imaging data analyzing modules;
storing the medical data in the database;
receiving a request for analysis of the imaging data from a user;
in response to the user request, analyzing the imaging data utilizing one of the plurality of modules to generate output data;
storing the output data in the database; and
providing at least one of the imaging data and the output data to a remote requester based on the at least one identifier.

2. The method according to claim 1, wherein the imaging data includes at least one of radiology data, radiology images, pathology image data, photographic images, scanned images, molecular imaging data and medical genetic imaging data.

3. The method according to claim 1, wherein the at least one identifier includes at least one of an identifier identifying the data analysis facility, an identifier identifying a patient from whom the medical imaging data was obtained and access data code indicating access parameters for the medical data

4. The method according to claim 1, wherein the imaging data is generated from a radiology procedure including at least one of Computerized Tomography scan, Magnetic Resonance Imaging, Positron Emission Technology, X-Rays, Vascular Interventional and Angiogram/Angiography procedures and Ultrasound imaging.

5. The method according to claim 1, further comprising the step of:
before the first receiving step, removing personal information pertaining to the patient from the at least one identifier.

6. The method according to claim 1, wherein the plurality of modules include at least one of a remote analysis of morphology features module, a nodule detection module for CXR scans, a nodule detection module for CT scans, an emphysema evaluation module, an airway evaluation module, a high-resolution data module, a growth assessment module, a volume determination module, a nodule detection module, a feature analysis module, a vascular analysis module, a two-dimensional analysis module, a three-dimensional analysis module, a one-dimensional analysis module and a time dependent four-dimensional module.

7. The method according to claim 1, wherein the data analysis facility is separate and independent of the medical facility.

8. The method according to claim 1, wherein the data analysis facility is responsible for obtaining and maintaining the analyzing modules.

9. The method according to claim 8, wherein the data analyzing facility provides access to utilization of the analyzing modules on a predetermined pricing structure.

10. The method according to claim 9, wherein the predetermined pricing structure includes at least one of a fixed monthly charge and a charge for each use of the selected analyzing module.

11. A system, comprising:
a remote server including a database, the remote server receiving, via a communications network, medical data from a medical facility and storing the medical data in the database, the medical data including at least one identifier and imaging data generated at the medical facility; and
a plurality of imaging data analyzing modules,
wherein the server receives a request for analysis of the imaging data from a user and wherein in response to the user request, the server controls one of the plurality of modules to analyze the imaging data to generate output data, the server storing the output data in the database, the server provides at least one of the imaging data and the output data to a remote requester based on the at least one identifier.

12. The system according to claim 11, wherein the imaging data includes at least one of radiology data, radiology images, pathology image data, photographic images, scanned images, molecular imaging data and medical genetic imaging data.

13. The system according to claim 11, wherein the at least one identifier includes at least one of an identifier identifying the data analysis facility, an identifier identifying a patient from whom the medical imaging data was obtained and access data code indicating access parameters for the medical data

14. The system according to claim 11, wherein the imaging data is generated from a radiology procedure including at least one of Computerized Tomography scan, Magnetic Resonance Imaging, Positron Emission Technology, X-Rays, Vascular Interventional and Angiogram/Angiography procedures and Ultrasound imaging.

15. The system according to claim 11, wherein personal information pertaining to the patient is removed from the at least one identifier before the medical data is received by the server.

16. The system according to claim 11, wherein the plurality of modules include at least one of a remote analysis of morphology features module, a nodule detection module for CXR scans, a nodule detection module for CT scans, an emphysema evaluation module, an airway evaluation module, a high-resolution data module, a growth assessment module, a volume determination module, a nodule detection module, a feature analysis module, a vascular analysis module, a two-dimensional analysis module, a three-dimensional analysis module, a one-dimensional analysis module and a time dependent four-dimensional module.

17. The system according to claim 11, wherein the system is separate and independent of the medical facility.

18. The system according to claim 11, wherein the system is responsible for obtaining and maintaining the analyzing modules.

19. The system according to claim 18, wherein the system provides access to utilization of the analyzing modules on a predetermined pricing structure.

20. The system according to claim 19, wherein the predetermined pricing structure includes at least one of a fixed monthly charge and a charge for each use of the selected analyzing module.

21. A method for remote analysis of medical imaging data, comprising the steps of:
generating medical imaging data of a patient at a medical facility;
tagging the medical imaging data with a first identifier identifying the medical facility, a second identifier identifying the patient and an access data indicative of access parameters for the medical imaging data;
transmitting the tagged medical imaging data to a remote data analysis facility;
storing the tagged medical imaging data at the data analysis facility;
receiving a request for analysis of the data from a user;
operating a selected one of a plurality of modules to analyze the medical imaging data based on the user request; and
providing at least one of the medical imaging data and the output data to a remote requester based on the access data.
